(19) United States
(12) Patent Application Publication
(10) Pub. No.: US 2012/0021022 A1
(43) Pub. Date: Jan. 26, 2012

(54) FOAMABLE SHAVING SUBSTRATE

(76) Inventor: Edgar Hakim, West Hollywood, CA (US)

(21) Appl. No.: 12/986,964

(22) Filed: Jan. 7, 2011

Related U.S. Application Data
(60) Provisional application No. 61/335,482, filed on Jan. 8, 2010.

(51) Int. Cl. A61K 8/02; A61K 8/72; A61Q 9/02; A61K 8/00

(52) U.S. Cl. 424/401; 424/73; 424/70.11

(57) ABSTRACT

The present invention provides a personal care product in the form of a foamable shaving substrate that includes a dry substrate that has associated with it a dry shaving material. The shaving material forms a moist hair softening foam when the substrate is exposed to water, facilitating shaving of hair. The foamable shaving substrate may easily be carried anywhere.

### CROSS-REFERENCE TO RELATED APPLICATIONS

This Application claims the benefit of priority of the co-pending U.S. Utility Provisional Patent Application No. 61/335,482, with a filing date of 8 Jan. 2010, the entire disclosures of which is expressly incorporated by reference in its entirety herein.

### BACKGROUND OF THE INVENTION

1. Field of the Invention

This invention is related to a personal care product in general and, more particularly, is related to a foamable shaving towel that can simulate and provide the comfort of a barbershop hot towel shave to prepare skin for smooth, soothing, and pleasant shaving experience.

2. Description of Related Art

In general, a barbershop hot towel shave includes the step of preparing the facial hair for shaving by application of hot, moist towel. The heated moisture from the hot, moist towel softens the facial hair, facilitating smooth, soothing, and pleasant shaving experience. The hot, moist towel is wrapped and pressed against the facial skin to heat and moisten facial hair, which results in softer hair for a smoother shave. Thereafter, the towel is removed, and a separate shaving product such as a shaving cream or gel that is generally maintained at a normal room temperature is applied onto the facial skin for shaving. Regrettably, an obvious disadvantage in a conventional barbershop hot towel shave is that once the towel is removed, the facial skin cools. In fact, the facial skin cools even further when a shaving cream or gel that is usually maintained at the room temperature is then applied onto the skin. The quick change in heating and then cooling of the skin is generally not pleasant, and the cooled facial hair may in fact loose its softness, which defeats the purpose and affect of applying hot moist towel to the skin.

Despite the drawbacks of a barbershop hot towel shave, in general, it is very difficult to have the same pleasant experience of shaving at home or when traveling as one received at a barbershop. Most other conventional shaving mechanisms simply require water, a shaving cream or gel, and a blade (without the use of hot, moist towels). A typical shave at home, travel, or camp involves the direct application of warm water (if any) to soften hair, any well-known shaving cream or gel to facilitate a smooth shave, and the use of a blade to shave off skin hair. Of course, it would be difficult (if not impossible) to have the same experience of a barbershop hot towel shave when at home, traveling, or camping. In fact, it is generally inconvenient to carry a container with a separate shaving material when traveling or camping, and most likely not allowed when traveling by air due to airport security. Conventional shaving cream or gels are usually in a container that is burdensome for travel (e.g., passing a security check with liquid volume limitation).

Other products exist that combine a very small, pocket size mitt like cloth (the size of which is specifically made to barely fit the index and middle finger of a user) combined with a dry shaving gel that is activated when the mitt is moistened. However, such products are not sufficiently large to provide the barbershop hot towel experience or to provide ease of application for larger skin areas (such as legs), and do not and are not made to foam.

Accordingly, in light of the current state of the art and the drawbacks to current shaving mechanisms, a need exists for delivery of shaving material that would provide the experience of having a barbershop hot towel shave anywhere, including home, when traveling, or camping, but without the drawbacks of the barbershop hot towel shaving mentioned above.

### BRIEF SUMMARY OF THE INVENTION

The present invention provides a method for delivery of shaving material, comprising:

associating dry shaving material with a dry substrate;

presenting water to the dry substrate for converting the associated dry shaving material into a moist shaving foam that includes hair softener for shaving.

Another optional exemplary aspect of the present invention provides a method for delivery of shaving material, wherein:

the shaving material is combined with the substrate.

Still another optional exemplary aspect of the present invention provides a method for delivery of shaving material, wherein:

the substrate contains the shaving material.

Yet another optional exemplary aspect of the present invention provides a method for delivery of shaving material, wherein:

the substrate is associated with the shaving material by adhesion.

A further optional exemplary aspect of the present invention provides a method for delivery of shaving material, wherein:

the substrate is associated with the shaving material by impregnation.

Still a further optional exemplary aspect of the present invention provides a method for delivery of shaving material, wherein:

the substrate is one of a towel, paper, and a polymer.

Another optional exemplary aspect of the present invention provides a method for delivery of shaving material, wherein:

the substrate is a porous material.

Another aspect of the present invention provides a shaving product, comprising:

a dry substrate;

dry shaving material associated with the dry substrate;

wherein the shaving material associated with the substrate forms a moist hair softening foam in presence of water, facilitating shaving of hair.

Another optional exemplary aspect of the present invention provides a shaving product, wherein:

the substrate is associated with the shaving material by adhesion.

Still another optional exemplary aspect of the present invention provides a shaving product, wherein:

the substrate is associated with the shaving material by impregnation.

Yet another optional exemplary aspect of the present invention provides a shaving product, wherein:

the substrate is one of a towel, paper, and a polymer.

A further optional exemplary aspect of the present invention provides a shaving product, wherein:

the substrate is a porous material.

Still a further optional exemplary aspect of the present invention provides a shaving product, wherein:

the shaving material is combined with the substrate.

Yet a further optional exemplary aspect of the present invention provides a shaving product, wherein:

the substrate contains the shaving material.

Another aspect of the present invention provides a shaving product, comprising:

a dry substrate;

dry shaving material associated with the dry substrate;

wherein the shaving material associated with the substrate forms a heated hair softening foam in presence of heated water, delivering simulated comfortable hot towel barbershop shave.

Such stated advantages of the invention are only examples and should not be construed as limiting the present invention. These and other features, aspects, and advantages of the invention will be apparent to those skilled in the art from the following detailed description of preferred non-limiting exemplary embodiments, taken together with the drawings and the claims that follow.

### BRIEF DESCRIPTION OF THE DRAWINGS

It is to be understood that the drawings are to be used for the purposes of exemplary illustration only and not as a definition of the limits of the invention. Throughout the disclosure, the word “exemplary” is used exclusively to mean “serving as an example, instance, or illustration.” Any embodiment described as “exemplary” is not necessarily to be construed as preferred or advantageous over other embodiments.

Referring to the drawings in which like reference character(s) present corresponding part(s) throughout:

FIG. 1 is an exemplary illustration of a foamable shaving substrate in accordance with the present invention.

### DETAILED DESCRIPTION OF THE INVENTION

The detailed description set forth below in connection with the appended drawings is intended as a description of presently preferred embodiments of the invention and is not intended to represent the only forms in which the present invention may be constructed and or utilized.

The present invention simulates the experience of a barbershop hot towel shave anywhere, including home, when traveling, or camping, but without its drawbacks. The further advantages of the present invention include elimination of carrying a large container of shaving cream or gel, reduction of shaving material volume, easiness of storage, portable, and suitable for all types of travel.

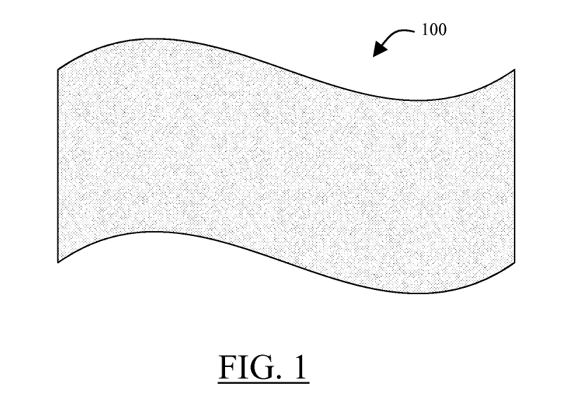

FIG. 1 is an exemplary illustration of a foamable shaving substrate in accordance with the present invention. As illustrated, the present invention provides a dry substrate 100 that has associated therewith a dry shaving material, wherein the shaving material forms a moist hair softening shaving foam or cream in present of water, facilitating shaving of hair. The substrate may comprise of a towel, paper, porous material, a polymer, or any combinations thereof with the shaving material combined with the substrate by adhesion onto the substrate, impregnation into the substrate, or other mechanisms or combinations thereof. The dry shaving material combined with the substrate will form a shaving foam or cream in the presents of liquid (e.g., water), which can be used for shaving. As illustrated, the foamable shaving substrate of the present invention is in sheet or strip shape (similar to a towel), dry or semi-dry, and therefore is easy to carry for travel. In addition, the shaving towel and or strip according to the present invention provides soothing comfort as if a hot towel is applied, in particular, in cold weather areas when used with hot water to activate the shaving material, simulating a barbershop hot towel shaving experience anywhere.

In general, the dry shaving material may be comprised of a chemical that is capable of absorbing water, expanding, and foaming from or lubricating gel in the presence of water (e.g., cold water or hot water). In absence of water, the dry shaving material is unexpanded in dry or semi-dry state combined with the substrate in shape that can be easily carried or stored.

The method of using the foamable shaving substrate of the present invention includes applying water onto the substrate, which activates the dry shaving material to foam, and applying the foam to skin for shaving. The term “skin” herein includes any skin areas of a body (e.g., the face, head, arm, legs, etc.) or a subject (e.g., an animal). The foamable shaving substrate when placed under hot water would form a warm foam or cream and when pressed against the face, and held thereon for a few seconds, would simulate a barbershop hot towel shaving experience with the added advantage of a warm activated residual foam on the skin is warm. Accordingly, the warm substrate is not removed to reduce skin temperature to cool skin hair, and the formed foam is not at room temperature, but is at the same temperature as that of the applied water. Therefore, the user does not experience a quick change in heating and then cooling of the skin, and the warm hair is not cooled to lose its softness. Once the skin is fully covered by the activated foam (a residual foam left on the skin), the substrate is set aside, and the user commences shaving to thereby fully simulate a barbershop hot towel shave anywhere.

Although the invention has been described in considerable detail in language specific to structural features and or method acts, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) is not used to show a serial or numerical limitation but instead is used to distinguish or identify the various members of the group.

In addition, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of,” “act of,” “operation of,” or “operational act of” in the claims herein is not intended to invoke the provisions of 35 U.S.C. 112, Paragraph 6.

What is claimed is:

1. A method for delivery of shaving material, comprising:
associating a dry shaving material with a dry substrate;
presenting water to the dry substrate for converting the associated dry shaving material into a moist shaving foam that includes hair softener for shaving.

2. The method for delivery of shaving material as set forth in claim 1, wherein:
the shaving material is combined with the substrate.

3. The method for delivery of shaving material as set forth in claim 1, wherein:
the substrate contains the shaving material.

4. The method for delivery of shaving material as set forth in claim 1, wherein
the substrate is associated with the shaving material by adhesion.

5. The method for delivery of shaving material as set forth in claim 1, wherein
the substrate is associated with the shaving material by impregnation.

6. The method for delivery of shaving material as set forth in claim 1, wherein:
the substrate is one of a towel, paper, and a polymer.

7. The method for delivery of shaving material as set forth in claim 1, wherein:
the substrate is a porous material.

8. A mechanism for shaving, comprising:
a dry substrate;
a dry shaving material associated with the dry substrate;
wherein the shaving material associated with the substrate forms a moist hair softening foam in presence of water, facilitating shaving of hair.

9. The mechanism for shaving as set forth in claim 6, wherein:
the substrate is associated with the shaving material by adhesion.

10. The mechanism for shaving as set forth in claim 6, wherein:
the substrate is associated with the shaving material by impregnation.

11. The mechanism for shaving as set forth in claim 6, wherein:
the substrate is one of a towel, paper, and a polymer.

12. The mechanism for shaving as set forth in claim 6, wherein:
the substrate is a porous material.

13. The mechanism for shaving as set forth in claim 6, wherein:
the shaving material is combined with the substrate.

14. The mechanism for shaving as set forth in claim 6, wherein:
the substrate contains the shaving material.

15. A mechanism for shaving, comprising:
a dry substrate;
a dry shaving material associated with the dry substrate;
wherein the shaving material associated with the substrate forms a heated hair softening foam in presence of heated water, delivering simulated comfortable hot towel barbershop shave.
